(19) United States
(12) Patent Application Publication
(10) Pub. No.: US 2014/0155973 A1
(43) Pub. Date: Jun. 5, 2014

(54) TISSUE-CAPTURED ANCHORS AND METHODS OF USE

(71) Applicant: Spinal Modulation, Inc., Menlo Park, CA (US)

(72) Inventors: Eric Grigsby, Napa, CA (US); Albert Burdulis, San Francisco, CA (US); Fred Linker, Los Altos, CA (US); Evan Vandenbrink, San Francisco, CA (US); Jeffrey J. Lee, San Ramon, CA (US)

(21) Appl. No.: 13/827,356

(22) Filed: Mar. 14, 2013

Related U.S. Application Data
(60) Provisional application No. 61/733,800, filed on Dec. 5, 2012.

(51) Int. Cl. A61N 1/05

(52) U.S. Cl. CPC A61N 1/0558; A61N 1/05
USPC 607/117; 607/116

(57) ABSTRACT

Devices, systems and methods are provided for anchoring implantable medical devices to maintain an implanted position. In some embodiments, the medical devices are stimulation leads which are implanted near a portion of the neural anatomy for providing stimulation thereto. To maintain position of the lead, the lead is anchored with the use of a tissue-captured anchor which is attached to the lead at a desired point of anchoring. The anchor maintains position of the lead by resisting movement of the anchor between tissue layers at the point of anchoring.

### CROSS-REFERENCES TO RELATED APPLICATIONS

This application claims priority under 35 U.S.C. 119(e) to U.S. Provisional Patent Application No. 61/733,800, entitled “Tissue-Captured Anchors and Methods of Use”, filed on Dec. 5, 2012, which is incorporated herein by reference.

### STATEMENT AS TO RIGHTS TO INVENTIONS MADE UNDER FEDERALLY SPONSORED RESEARCH AND DEVELOPMENT

NOT APPLICABLE

### REFERENCE TO A “SEQUENCE LISTING,” A TABLE, OR A COMPUTER PROGRAM LISTING APPENDIX SUBMITTED ON A COMPACT DISK

NOT APPLICABLE

### BACKGROUND OF THE INVENTION

Electrical stimulation and drug delivery to portions of the anatomy, particularly the spinal anatomy and peripheral nervous system, often involve the implantation of one or more leads or delivery devices within the patient's body. The leads or delivery devices extend between the target anatomy and an implantable pulse generator (IPG) or drug reservoir which is typically implanted at a remote location. Precise positioning of the leads or delivery devices is desired to optimize treatment. Accuracy in administration of the drugs or stimulation to a particular target location can maximize beneficial effects of treatment and patient satisfaction. It is desired that such accuracy be maintained over time to ensure continued successful treatment.

For example, when implanting an epidural lead, a physician must surgically open the body tissue to the epidural space, and then insert the lead into the epidural space to the desired location. Fluoroscopy aids the physician, and trial and error tests of treatment define the desired location(s) for treatment. Once desirably positioned, it is desired to maintain the lead in place. Typically this is attempted by suturing the lead in place, such as by attaching a sleeve to the lead and suturing the sleeve to the surrounding tissue where the lead enters the epidural space. In addition, sutures are placed to prevent movement between the sleeve and the lead. The quality of the connection between the sleeve, the lead and the surrounding tissue is often highly variable and depends on the tightness of the sutures or other attachment means. Such suturing is time consuming, tedious and subject to error or variability. Further, any repositioning requires removal of the sutures and resuturing. Also, such suturing is dependent on the quality and availability of suitable surrounding tissue and accessibility to the physician.

It is desired to provide mechanisms for anchoring leads, catheters or other devices within body tissue that are easy and efficient to use, reliable, and adjustable. At least some of these objectives will be met by the present invention.

### SUMMARY OF THE INVENTION

Aspects of the present disclosure provide devices, systems, and methods for anchoring implantable medical devices to maintain an implanted position.

In a first aspect of the present invention, a tissue-captured anchor is provide for anchoring an elongate device within a body of a patient. In some embodiments, the anchor comprises an anchor body suturelessly attachable to the elongate device at an anchoring point and contoured so as to a) be positionable between a first tissue layer and a second tissue layer within the body while the elongate device passes through the first tissue layer and the second tissue layer, and b) atraumatically resist movement through the tissue layers thereby anchoring the elongate device between the tissues at the anchoring point. In some embodiments, the contour has a ball, round, elliptical, oval, oblong or disk shape. Typically, the anchor has a diameter of less than 0.5 inches. In some embodiments, the anchor is sized and contoured to be passable through an incision in a muscle or ligament, wherein the incision has a length of less than 1 inch. In some embodiments, the contour includes at least one protruding portion which extends laterally outward from the elongate device.

In some embodiments, the anchor body comprises a first portion having first lumen configured for passage of the elongate device therethrough and a second portion having a second lumen configured for passage of the elongate device therethrough, wherein the first and second lumens are alignable for passage of the elongate device therethrough. In such embodiments, a misalignment of the first and second lumens may cause attachment of the anchor body to the elongate device. For example, the first portion may comprise a plunger which is advanceable within the second portion so such advancement aligns or misaligns the first and second lumens. Optionally, alignment or misalignment may be maintained by force of a spring. In some embodiments, the first portion is moveable toward the second portion wherein such movement causes the lumens to misalign. In some embodiments, the first portion has a first mating surface the second portion has a second mating surface, wherein mating of the first and second surfaces together attaches the anchor to the elongate device. In some embodiments, the first portion has a protrusion through which the first lumen passes and the second portion has a recession through which the second lumen passes, wherein mating of the protrusion with the recession aligns the first and second lumens and attaches the anchor to the elongate device. In some embodiments, the first portion has a first perimeter and the second portion has a second perimeter wherein aligning or misaligning the perimeters attaches the anchor to the elongate device. In some embodiments, rotating the first portion in relation to the second portion attaches the anchor to the elongate device.

It may be appreciated that in some embodiments, the anchor body comprises a first mating surface and a second mating surface, wherein the first and second mating surfaces are mateable to each other while the elongate device is disposed therebetween. In some instances, the anchor body comprises a first portion having the first mating surface and a separate second section having the second mating portion, wherein the first and second portions are joinable. In some instances, the first mating surface is disposed on a first jaw and the second mating portion is disposed on a second jaw, wherein the jaws are connected on at least one side and open to receive the elongate device therebetween. For example, the anchor body may be configured so that squeezing an outer perimeter of the anchor body toward its center axis flexes and moves the first jaw away from the second jaw so that the surfaces un-mate. Or, the first and second jaws form a side opening in the anchor body for insertion of the elongate device therebetween.

In some embodiments, the anchor body has a lumen configured for passage of the elongate device therethrough and a cam arranged to at least partially obstruct the lumen so as to attach the anchor to the elongate device.

In some embodiments, the anchor body is removeably attachable to the elongate device.

In a second aspect of the present invention, a method is provided for anchoring an elongate device within a body of a patient. In some embodiments, the method comprises positioning an anchor between a first tissue layer and an adjacent second tissue layer within the body; and suturelessly attaching the anchor to an elongate device at an anchoring point, wherein the elongate device is positioned through the first tissue layer and second tissue layer within the body and wherein the anchor is contoured to atraumatically resist movement through the tissue layers thereby anchoring the elongate device between the tissues at the anchoring point.

In some embodiments, positioning comprises positioning the anchor laterally adjacent to a spinous process. For example, first tissue layer may be comprised of a spinous muscle layer. Optionally, the second tissue layer may also be comprised of a spinous muscle layer.

In some embodiments, the first layer or second layer comprises fascia, a spinae erector, an illiocostalis lumborum, a longissimus thoriclis, a longissimus cervicus, an illioconstalis cervicis, a serratus anterior, a ligament, a supraspinous ligament, an interspinous ligament, a ligamentum flavum, an alar ligament, an anterior atlantoaxial ligament, a posterior atlantoaxial ligament, a ligamentum nuchae, an anterior longitudinal ligament, a posterior longitudinal ligament, an interspinous ligament, an intertransverse ligament, an iliolumbar ligament, a sacroiliac ligament, a sacrospinous ligament, a sacrotuberous ligament, an anterior occipitoatlantal ligament, a posterior occipitoatlantal ligament, a lateral occipitoatlantal ligament, an occipitoaxial ligament, an apical ligament, an altantoaxial ligament, a lateral ligament, a transverse ligament, a superior longitudinal fascicle, an inferior longitudinal fascicle, an aponeurosis, a tendon, a subcutaneous tissue, skin, a dermal layer, a bone, cartilage, or an artificial tissue.

In some embodiments, the anchor body comprises a first portion having a first lumen configured for passage of the elongate device therethrough and a second portion having a second lumen configured for passage of the elongate device therethrough, the method further comprising mounting the anchor on the elongate device by passing the elongate device through the first and second lumens while the lumens are aligned. In some embodiments, suturelessly attaching the anchor to the elongate device comprises misaligning the lumens. In some embodiments, suturelessly attaching the anchor to the elongate device comprises rotating the first portion in relation to the second portion.

In some embodiments, the first portion has a first mating surface and the second portion has a second mating surface, wherein suturelessly attaching the anchor to the elongate device comprises mating the first and second surfaces together.

In some embodiments, the first portion has a first perimeter and the second portion has a second perimeter, wherein suturelessly attaching the anchor to the elongate device comprises aligning or misaligning the perimeters.

In some embodiments, the anchor body comprises a first mating surface and a second mating surface, wherein suturelessly attaching the anchor to the elongate device comprises mating the first and second mating surfaces to each other while the elongate device is disposed therebetween. For example, when the first mating surface is disposed on a first jaw and the second mating surface is disposed on a second jaw, suturelessly attaching the anchor to the elongate device may comprise opening the jaws to receive the elongate device therebetween. Optionally, opening the jaws comprises squeezing an outer perimeter of the anchor body toward its center axis which causes the first jaw to move away from the second jaw. Or, wherein the first and second jaws form a side opening in the anchor body, suturelessly attaching the anchor to the elongate device may comprise inserting the elongate device into the side opening.

In some embodiments, the method further comprises releasing the attachment of the anchor to the elongate device.

In other embodiments, the method, further comprising suturelessly re-attaching the anchor to the elongate device.

Other objects and advantages of the present invention will become apparent from the detailed description to follow, together with the accompanying drawings.

### BRIEF DESCRIPTION OF THE DRAWINGS

FIG. 1 illustrates an example of such an implantable lead advanced into the epidural space.

FIG. 2A illustrates an embodiment of a tissue-captured anchor at an example anchoring position within the anatomy.

FIG. 2B illustrates a cut-away side view of the spine with a lead and anchor positioned similarly to FIG. 2A.

FIG. 3 illustrates an embodiment of a tissue-captured anchor attached to a lead at a point along its length so that the anchor resides between a first tissue layer and a second tissue layer.

FIGS. 4A-4C illustrate one technique of positioning the anchor between the tissue layers.

FIGS. 5A-5B, 6A-6B, 7 illustrate an embodiment of a split-barrel anchor.

FIGS. 8-9 illustrate an embodiment of a wave anchor.

FIGS. 10, 11, 12 illustrate another embodiment of a wave anchor.

FIGS. 13, 14, 15, 16 illustrate an embodiment of a disk anchor.

FIGS. 17, 18, 19 illustrate an embodiment of a plunger anchor.

FIGS. 20A-20B, 21, 22 illustrate an embodiment of a cam anchor.

FIGS. 23A-23B, 24A-24B illustrate embodiments of a jaw anchor.

FIGS. 25, 26A-26B illustrates an embodiment of a flapper anchor.

FIGS. 27A-27B illustrate an embodiment of a balloon anchor.

FIGS. 28A-28B illustrate an embodiment of an umbrella anchor.

FIGS. 29A-29B illustrate an embodiment of a twist-grip anchor.

FIG. 30 illustrates an embodiment of a closure device.

FIGS. 31A-31B illustrate an embodiment of a locking device used with the closure device of FIG. 30.

### DETAILED DESCRIPTION OF THE INVENTION

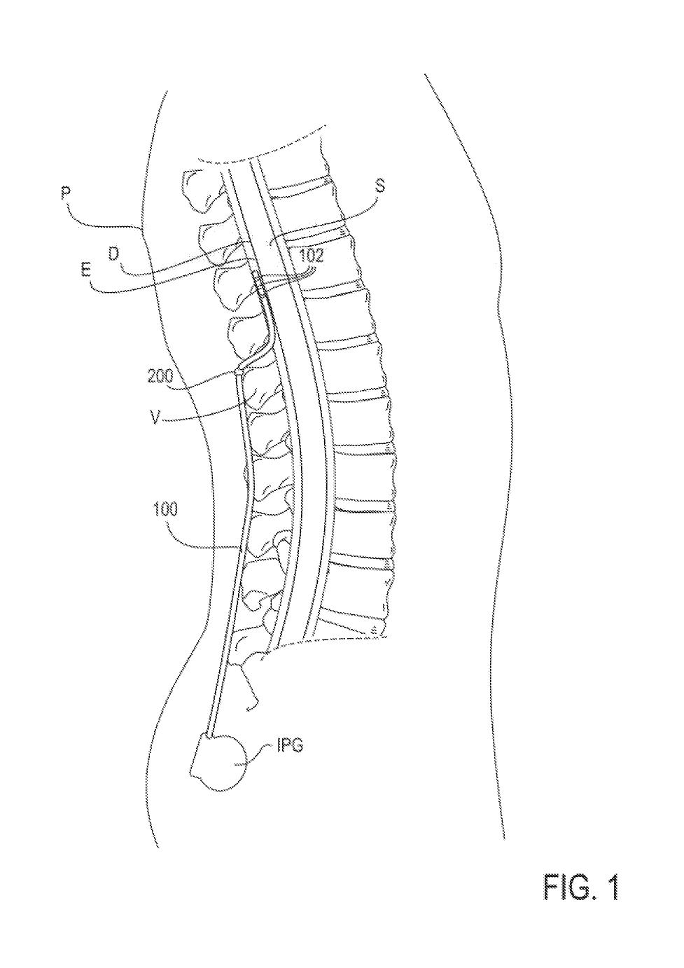

The present invention provides devices, systems and methods for anchoring implantable medical devices to maintain an implanted position. In some embodiments, the medical devices are stimulation leads which are implanted near a portion of the neural anatomy for providing stimulation thereto. In some embodiments, at least one lead is advanced into the epidural space to apply stimulation energy to the spinal cord itself or to anatomies accessible via the epidural space, such as the dorsal root, dorsal root ganglion or peripheral nerves. FIG. 1 illustrates an example of such an implantable lead 100 advanced into the epidural space E. Here, the lead 100 is shown inserted between the vertebrae V, advanced within the epidural space E and positioned so that electrodes 102 disposed along its distal end are positioned against the dura layer of the spinal cord S. It may be appreciated that the lead 100 may be advanced further, such as to position the electrodes 102 near other spinal anatomy, such as the dorsal root ganglion. In any case, the lead 100 is implanted either through the skin via an epidural needle or through an open procedure involving a cut-down to the desired anatomy. The leads 100 extend from the epidural space E to an implantable pulse generator IPG which is implanted at a remote location, such as in the buttocks. To maintain position of the lead 100, the lead 100 is anchored with the use of a tissue-captured anchor 200 which is attached to the lead 100 at a desired point of anchoring. The anchor 200 maintains position of the lead 100 by resisting movement of the anchor 200 between tissue layers at the point of anchoring.

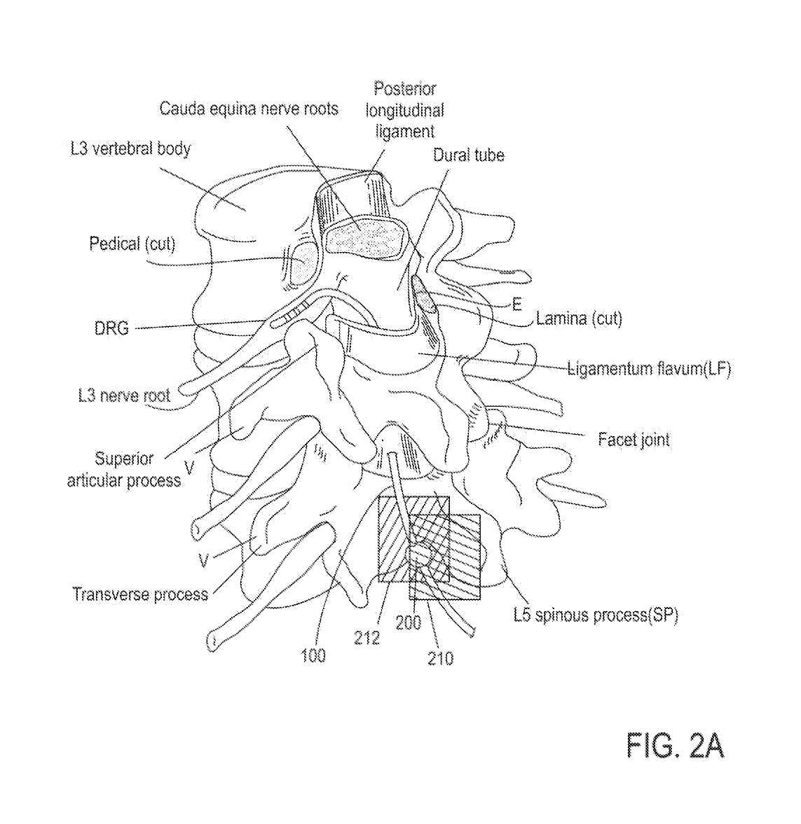

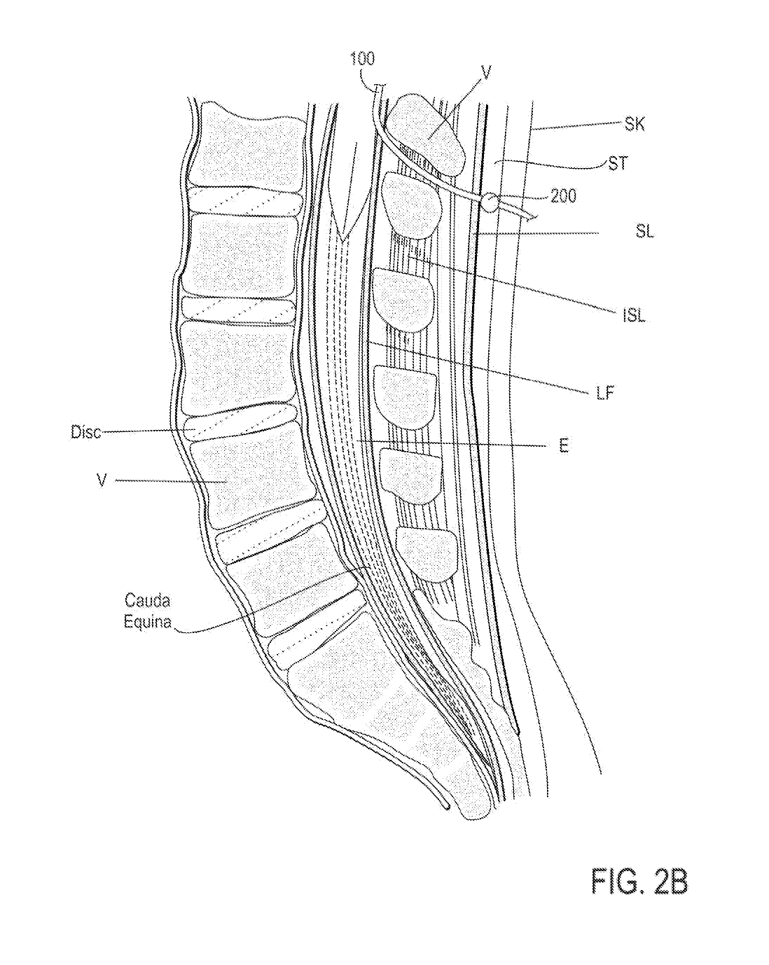

FIG. 2A illustrates an embodiment of a tissue-captured anchor 200 at an example anchoring position within the anatomy. Here, the distal end of the lead 100 is shown advanced between vertebrae V into the epidural space E so that one or more of the electrodes 102 are positioned on, near, about, adjacent or in proximity to the dorsal root ganglion DRG. The epidural space E can be accessed with the use of an introducing needle. Typically, the skin is infiltrated with local anesthetic such as lidocaine over the identified portion of the epidural space. Typically, the needle is inserted to the ligamentum flavum LF and a loss of resistance to injection technique is used to identify the epidural space. For such a technique, a syringe is attached to the needle. The syringe may contain air or saline. Traditionally either air or saline has been used for identifying the epidural space, depending on personal preference. When the tip of the needle enters a space of negative or neutral pressure (such as the epidural space), there can be a “loss of resistance” and it will be possible to inject through the syringe. At that point, there may now be a high likelihood that the tip of the needle has entered the epidural space. Further, a sensation of “pop” or “click” may be felt as the needle breaches the ligamentum flavum just before entering the epidural space. In addition to the loss of resistance technique, real-time observation of the advancing needle may be achieved with a portable ultrasound scanner or with fluoroscopy. Once the needle has been successfully inserted into the epidural space E, the syringe can be removed. The lead 100 is then delivered through the needle with the use of various delivery devices, such as described and illustrated in U.S. patent application Ser. No. 12/687,737, entitled “Stimulation Leads, Delivery Systems and Methods of Use”, filed Jan. 14, 2010, and incorporated by reference for all purposes.

A tissue-captured anchor 200 is shown attached to the lead 100 at a position or anchoring point along the elongate body of the lead 100 so that the anchor 200 resides outside of the ligamentum flavum LF. In this embodiment, the anchor 200 is positioned laterally adjacent to a spinous process SP, near the point of entry to the epidural space E. A plurality of spinous muscle layers reside along the back, adjacent to the spinous processes SP and portions of the vertebrae V. FIG. 2A schematically illustrates two spinous muscle layers (a first tissue layer 210 and a second tissue layer 212) between which the anchor 200 is positionable. Example spinous muscle layers include spinae erector, illiocostalis lumborum, longissimus thoriclis, longissimus cervicus, illioconstalis cervicis, and serratus anterior, to name a few. The anchor 200 is shaped or contoured so that the tissue layers 210, 212 resist movement of the anchor 200 in either direction or passage therethrough, holding or maintaining the anchor 200 in the desired position thus creating a point of anchoring. Typically, an incision is made through the tissue layer 210, 212 that is most dorsal and the anchor 200 is passed therethrough. When the incision is sutured closed, the tissue layers 210, 212 limit the movement of the anchor, holding the lead in place.

FIG. 2B illustrates a cut-away side view of the spine with a lead 100 and anchor 200 positioned similarly to FIG. 2A. As shown, the anchor 200 is attached to the lead 100 at a position or anchoring point along the elongate body of the lead 100 so that the anchor 200 resides outside of the ligamentum flavum LF. Again, the anchor 200 is positioned laterally adjacent to a spinous process SP, near the point of entry to the epidural space E. In this embodiment, the anchor 200 is shown as positioned in a plane dorsal to the suprasinous ligament SL. Such positioning may be desired to reduce any interference between the anchor 200 and the spinous process SP. However, in other embodiments, the anchor 200 is positioned adjacent the supraspinous ligament SL. In any case, the anchor 200 is typically nestled ventral to the dorsal-most fascia within the musculature dorsal to the ligamentum flavum LF. The anchor is situated such that when incision through the dorsal-most musculature or fascia is sutured closed, the tissue layers limit movement of the anchor 200 in either direction or passage therethrough, holding or maintaining the anchor in the desired position thus creating a point of anchoring.

Typically, the anchor 200 has a ball, round, oblong or disk shape so as to be atraumatic to the tissue and resist movement through tissue layers. In some embodiments, the anchor 200 has a diameter of less than 0.5 inches, particularly 0.2-0.4 inches. It may be appreciated that a variety of sizes may be used such that the anchor is small enough to reduce trauma to nearby tissues and minimize patient discomfort while being big enough to anchor the lead and allow for physician handling. In some embodiments, the anchor 200 has a dimension of up to 6 mm; examples of such a dimension include diameter of a disk shape or ball shape. Since such anchoring is achieved due to anchor shape, position of the anchor, and/or applying suturing to the tissue dorsal to the anchor, suturing of the anchor directly to the tissues is not needed. Suturing is used to close the incision through which the anchor 200 is passed. Such suturing to close the tissue over the anchor is typically achieved with one or two suture knots. Thus, the incision-closure sutures do not have to be uniformly tight or evenly tied, and only one or two sutures are required. These are advantages over conventional tissue anchors that are sutured to the device to which they are anchoring. Such conventional tissue anchors are typically sutured to the device or lead while closing the incision, thus making the steps interdependent. By eliminating suturing of the anchor to the lead, lead anchoring is much quicker and less tedious. And, anchoring is not subject to the suturing skills of the surgeon. In addition, if repositioning of the lead or anchoring point is desired, the tissue-captured anchor 200 is easily removed from the lead and repositioned without the need for removing sutures from the anchor and resuturing the anchor in place.

### Anchor Position

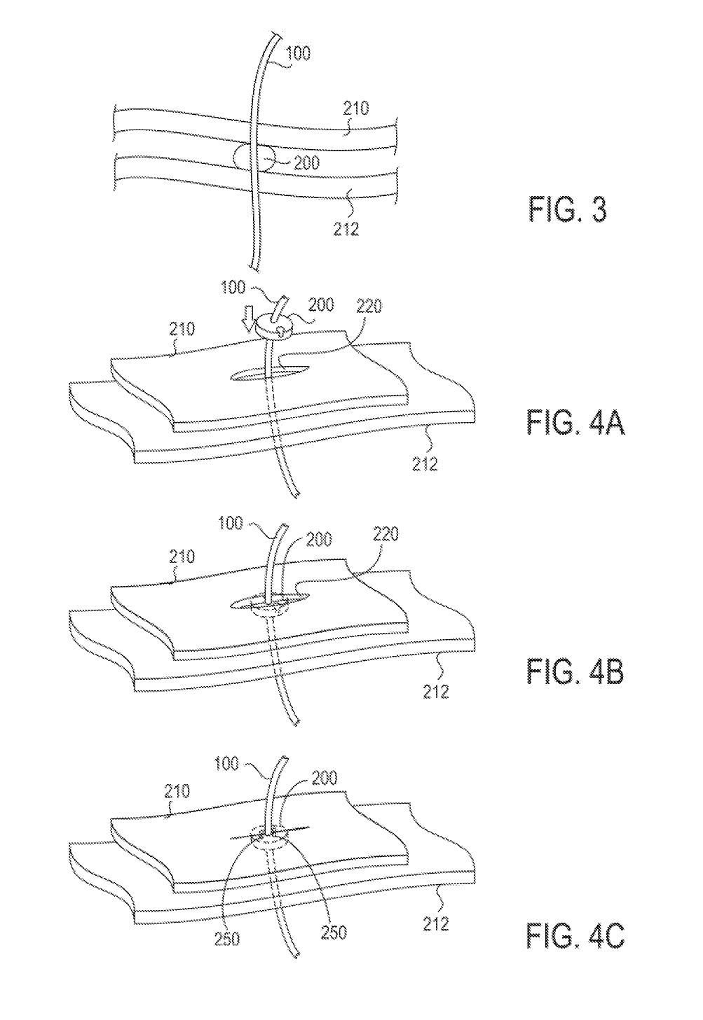

As illustrated in FIG. 3, the tissue-captured anchor 200 is attached to the lead 100 at a point along its length so that the anchor 200 resides between a first tissue layer 210 and a second tissue layer 212. FIGS. 4A-4C illustrate one technique of positioning the anchor 200 between the tissue layers 210, 212. In this embodiment, an incision 220 is made in the first tissue layer 210 through which a portion of the lead 100 passes, as illustrated in FIG. 4A. The anchor 200 is then inserted through the incision 220 and positioned between the first and second tissue layers 210, 212, as illustrated in FIG. 4B. In some embodiments, the anchor 200 is advanced along the lead 100 to the desired attachment position and in other embodiments the anchor 200 is simply positioned on the lead 100 at the desired attachment position. The anchor 200 is then fastened or fixedly secured to the lead 100. Referring to FIG. 4C, the incision 220 is then closed over the anchor 200, such as by sutures 250, allowing a portion of the lead 100 to protrude through the sutured incision 220. The anchor 200 is thus captured between the first and second tissue layers 210, 212 anchoring the lead 100 in place. Typically, the tissue layers 212 are comprised of strong tissues, such as fascia, ligaments or musculature layers. In some embodiments, the first tissue layer 210 comprises the spinous muscle layer and the second tissue layer 212 comprises an adjacent spinous muscle layer. It may be appreciated that the first and second tissue layers 210, 212 may be comprised of any combination of the following tissues or two of the same type of tissues: fascia, spinae erector, illiocostalis lumborum, longissimus thoriclis, longissimus cervicus, illioconstalis cervicis, serratus anterior, ligament, supraspinous ligament, interspinous ligament, ligamentum flavum, alar ligament, anterior atlantoaxial ligament, posterior atlantoaxial ligament, ligamentum nuchae, anterior longitudinal ligament, posterior longitudinal ligament, interspinous ligament, intertransverse ligament, iliolumbar ligament, sacroiliac ligament, sacrospinous ligament, sacrotuberous ligament, anterior occipitoatlantal ligament, posterior occipitoatlantal ligament, lateral occipitoatlantal ligament, occipitoaxial ligament, apical ligament, altantoaxial ligament, lateral ligaments, transverse ligaments, superior longitudinal fascicles, inferior longitudinal fascicles, aponeurosis, tendon, subcutaneous tissue, skin, and dermal layer, to name a few. It may also be appreciated that the first or second tissue layers 210, 212 may be comprised of bone or cartilage. It may also be appreciated that the first or second tissue layers 210, 212 may be comprised of artificial tissue layers or implant materials.

The anchor 200 is attached to the elongate body of the lead 100 and shaped to resist passage of the anchor through the holes in the tissue layers 210, 212 made by the lead 100. Thus, the anchor 200 is shaped to have at least one portion that broadens or widens the diameter of the lead body in a particular area to create resistance and a point of anchoring. In some embodiments, the anchor has a round, elliptical, pearl-like, oval, oblong or disk shape to name a few. In other embodiments, the anchor 200 has at least one protruding portion which extends in a direction perpendicular to the lead body or at an angle which impedes or resists passage of the anchor through the first tissue layer 210 and/or the second tissue layer 220. Thus, the anchor 200 is sandwiched between the first layer 210 and the second layer 212 to maintain position of the lead 100.

The anchor 200 is attached to the body of the lead 100 in a manner which fixes the anchor 200 in place so that it does not move along the lead body. This maintains position of the lead 100 while the anchor 200 is held by the tissues. In some embodiments, such attachment is achieved without the use of tools. Typically, such attachment is reversible so that the anchor 200 can be removed and repositioned along the lead 100 if desired. A variety of anchor 200 embodiments are provided herein.

### Split-Barrel Anchor

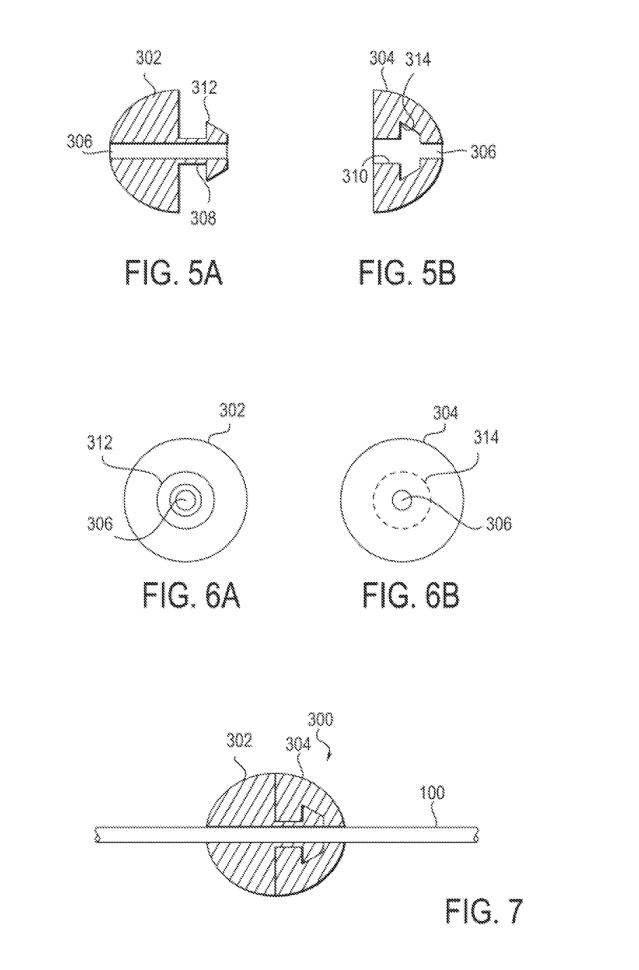

An embodiment of a split-barrel anchor 300 is illustrated in FIGS. 5A-5B, 6A-6B, 7. In this embodiment, the anchor 300 is comprised of a first portion 302 (FIG. 5A) and a second portion 304 (FIG. 5B) wherein the first and second portions 302, 304 mate to form a ball or sphere shape. Each portion 302, 304 has a lumen 306 for passage of a lead therethrough. When the portions 302, 304 are mated, the lumens align so that the anchor 300 can be advanced along the lead if desired. In this embodiment, the first portion 302 has a protrusion 308 and the second portion 304 has a recession 310 wherein the protrusion 308 mates with the recession 310. In this embodiment, the protrusion 308 has a flange 312 near its distal end which mates with an undercut 314 in the recession 310. Thus, at least the second portion 304 is sufficiently flexible to allow advancement of the undercut 314 over the protrusion 308 so that the undercut 314 holds the protrusion 308 in place and resists unmating or disengagement of the first and section portions 302, 304. FIG. 6A illustrates an end view of the first portion 302 having a lumen 306 passing through the protrusion 308. FIG. 6B illustrates an end view of the second portion 304 also having a lumen 306 passing therethrough, wherein the undercut 314 is hidden from view. FIG. 7 illustrates a lead 100 passing through the mated first and second portions 302, 304 of the anchor 300. The anchor 300 is advanceable along the lead 100 to a desired location for anchoring within the patient's anatomy, between tissue layers. Once positioned at the desired location, the anchor is then fixedly attached to the lead 100. This is typically achieved without the use of additional tools. In this embodiment, advancing the undercut 314 over the protrusion 308 and flange 312 (i.e. snapping the halves of the anchor together) deforms the inner diameter of the protrusion 308 so that it compresses against the lead body passing therethrough. This increases sliding friction between the lead 100 and the anchor 300 so that movement of the lead 100 is resisted. The anchor 300 may be removeable from the lead 100 in a variety of ways. In some embodiments, the anchor 300 is comprised of a material that is flexible enough to allow the protrusion 308 to be unsnapped or disengaged from the flange 312 by pulling the first and second portions 302, 304 apart. In other embodiments, a release tool may be used. Such a tool may include a plunger that engages the protrusion through the second portion 304, wherein it releases the flange 312 from the undercut 314. In such embodiments, the protrusion may extend through the recession 310 so that it is accessible through the second portion 304, such as adjacent the lumen through which the lead 100 passes.

### Wave Anchor

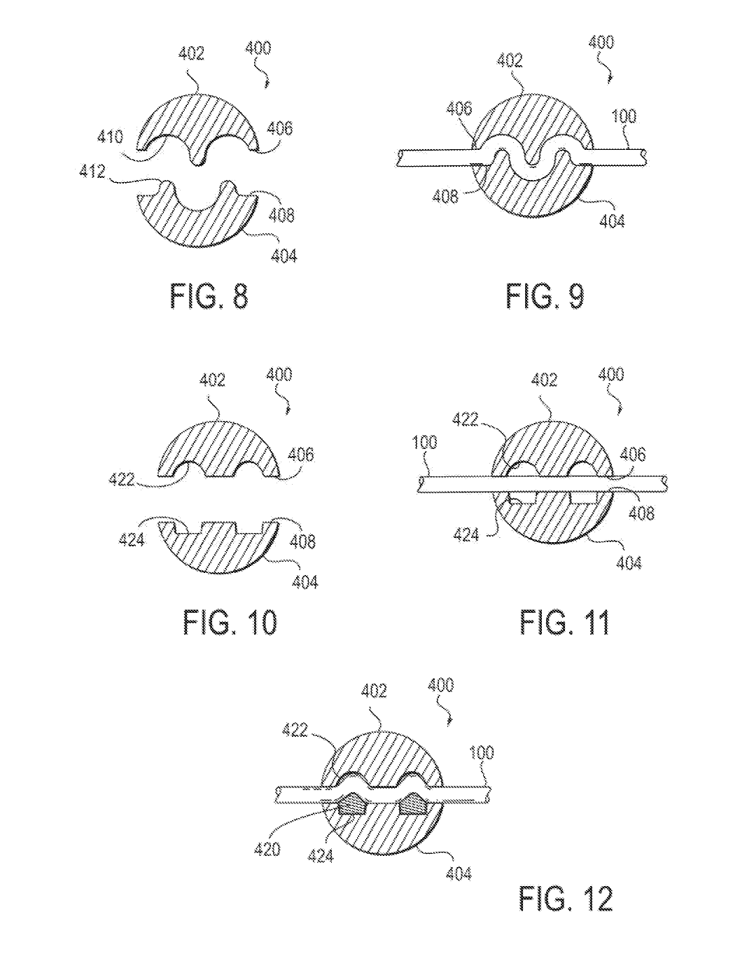

FIGS. 8-9 illustrate an embodiment of a wave anchor 400. In this embodiment, the anchor 400 is comprised of a first portion 402 and a second portion 404 wherein the first and second portions 402, 404 mate to form a ball or sphere shape. The first portion 402 has a first mating surface 406. The second portion 404 has a second mating surface 408. The mating surfaces 406, 408 are configured to fixedly hold a portion of a lead 100 therebetween when the first and second portions 402, 404 are mated to form the wave anchor 400. Typically, the lead 100 is held by friction. In some embodiments, the first mating surface 406 and/or second mating surface 408 has surface characteristics, such as curves, channels, waves, steps, grips or irregularities which increases the friction. In some embodiments the surface characteristics are matched or coordinated between the surfaces 406, 408. FIG. 8 illustrates the first mating surface 406 having indents 410 which match protrusions 412 of the second mating surface 408. Thus, when a lead 100 is positioned between the surfaces 406, 408 (FIG. 9), the lead 100 is conformed to the surface characteristics (i.e. the lead 100 is pressed into the indents by the protrusions creating friction that resists pull out of the lead). In some embodiments, the mated first and second portions 402, 404 are held in the mated configuration by latching or a retention mechanism such as a screw, barb, snap, lever arm, suture or a combination of these.

FIGS. 10, 11, 12 illustrate another embodiment of a wave anchor 400. In this embodiment, the anchor 400 is comprised of a first portion 402 and a second portion 404 wherein the first and second portions 402, 404 mate to form a ball or sphere shape. The first portion 402 has a first mating surface 406. The second portion 404 has a second mating surface 408. In this embodiment, the mating surfaces 406, 408 are configured to fixedly hold a portion of a lead 100 therebetween when the first and second portions 402, 404 are mated and inserts 420 are positioned to between the mating surfaces 406, 408 to assist in holding the lead 100 in place. For example, FIG. 10 illustrates an embodiment wherein the first mating surface 406 has indents 422 which align with indents 424 or channels of the second mating surface 408. When a lead 100 is positioned between the surfaces 406, 408 (FIG. 11), the lead 100 can be moved relative to the anchor 400. FIG. 12 illustrates the addition of inserts 420 which are positioned within the indents 424 of the second mating surface 408. The inserts 424 are larger than the indents 424 and therefore press the lead 100 against the indents 422 of the first mating surface 406 creating sufficient friction to resist pull-out of the lead. The inserts 420 can be positioned by hand or with the use of a tool. Again, the mated first and second portions 402, 404 are held in the mated configuration by latching or a retention mechanism such as a screw, snap, lever arm, suture or a combination of these.

### Disk Anchor

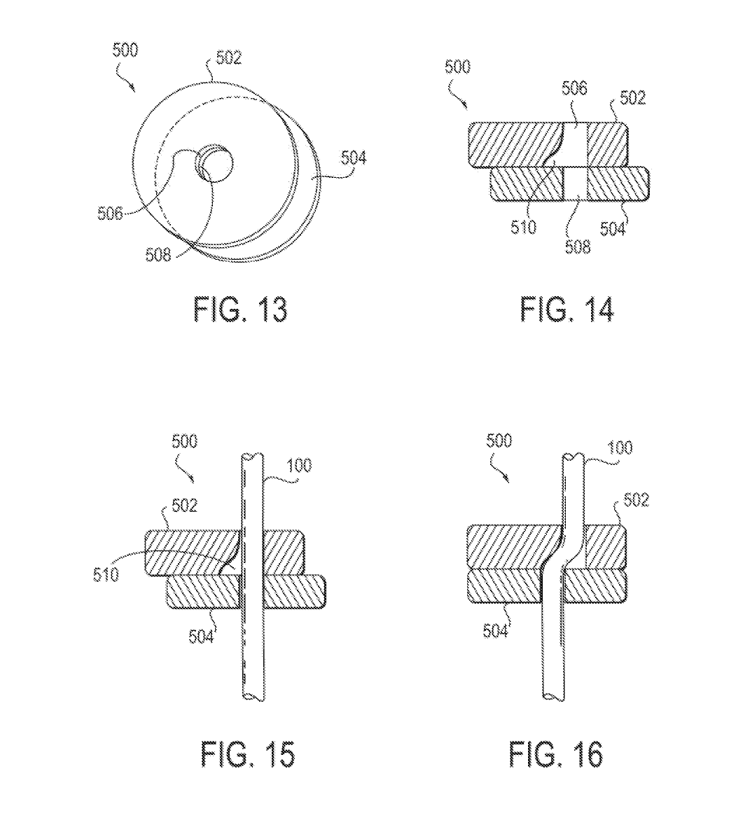

FIGS. 13, 14, 15, 16 illustrate an embodiment of a disk anchor 500. In this embodiment, the anchor 500 has a disk shape and is comprised of a first portion 502 and a second portion 504. The first portion 502 includes a first lumen 506 for passage of a lead therethrough and the second portion 504 also includes a second lumen 508 for passage of the lead therethrough. The anchor 500 is configured to be switchable between an unlocked position, wherein the lumens 506, 508 are aligned and locked position wherein the lumens 506, 508 are not aligned, misaligned, or off-set. The locked position fixedly holds the lead in relation to the anchor due to the tortuous path of the lead created by the off-set lumens 506, 508. FIG. 13 provides a top view illustration of the disk anchor 500 in the unlocked position. Here the lumens 506, 508 are aligned. In this embodiment, the first portion 502 and second portion 504 each have a disk shape, however the second lumen 508 is not concentric with the second portion 504 so the perimeters of the portions 502, 504 do not align when in the unlocked position. This aids the user in determining that the anchor 500 is unlocked. FIG. 14 provides a side-view illustration of the embodiment of FIG. 13. Thus, the lumens 506, 508 are shown as aligned while the perimeters or outside edges of the portions 502, 504 are off-set. The first portion 502 also includes a cut-out 510 which provides bending room for the lead during switching of the anchor 500 to the locked position. FIG. 15 illustrates a lead 100 advanced through the lumens 506, 508 while the anchor 500 is in the unlocked position. This position allows the anchor 500 to be moved along the lead 100 to a desired location. The anchor 500 can then be switched to the locked position by moving the first and second portions 502, 504 relative to each other, as indicated by arrows. In this embodiment, such movement aligns the perimeters or outside edges of the first and second portions 502, 504 and misaligns or off-sets the lumens 506, 508, as illustrated in FIG. 16. This action shifts, curves or bends the lead 100 so that the lead 100 is maintained in place by friction. In this embodiment, the lead 100 wraps against a luminal cut-out 510 which provides extra bending room so that the lead 100 resists kinking when in the anchor 500 is in the locked position. In some embodiments, the first and second portions 502, 504 are maintained in the locked position with the use of a locking mechanism, such as a peg sliding into a channel which has a protrusion for locking the peg therein.

### Plunger Anchor

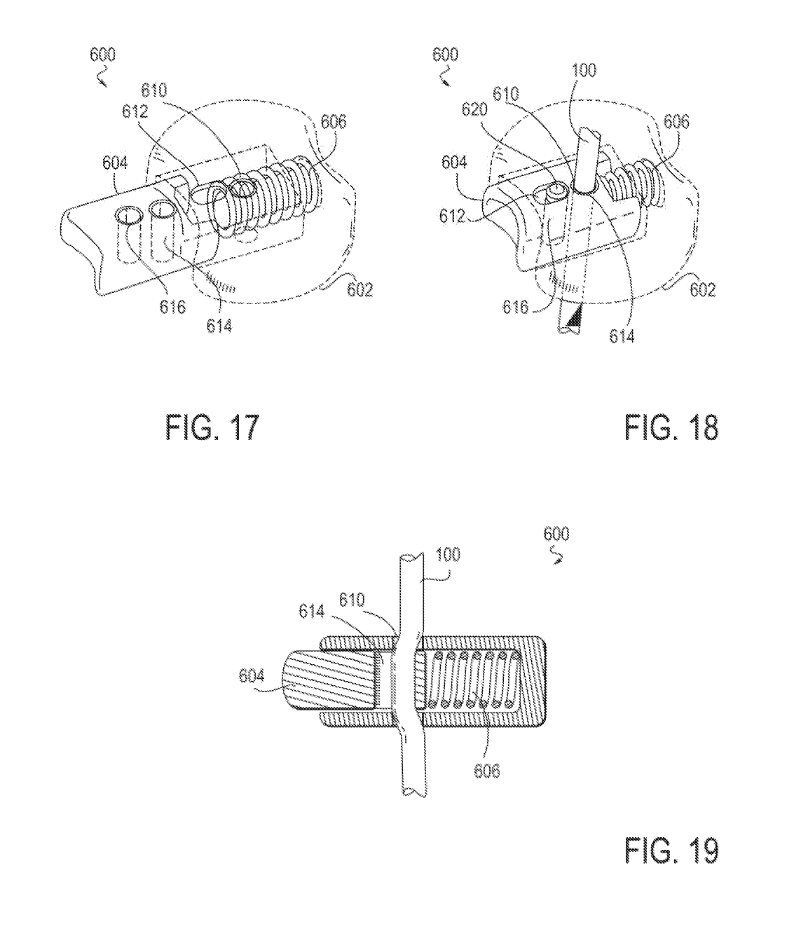

FIGS. 17, 18, 19 illustrate an embodiment of a plunger anchor 600. In this embodiment, the anchor 600 is comprised of an anchor body 602, a plunger 604 and a spring 606. In this embodiment, the anchor body 602 has a disk shape, however, it may be appreciated that the anchor body 602 can have any shape which resists movement of the anchor 600 through tissue layers. Referring to FIG. 17, the anchor body 602 includes a lead hole 610 and a locking hole 612. The plunger 604 includes a lead lumen 614 and a locking lumen 616. In a relaxed position (FIG. 17), the plunger 604, the holes 610, 612, and lumens 614, 616 are misaligned as the plunger 602 protrudes from the anchor body 602. Advancement of the plunger 602 into the anchor body 602, compresses the spring 606, as illustrated in FIG. 18. The plunger 602 may be advanced so that the lead hole 610 aligns with the lead lumen 614. Once aligned, a lead 100 can be advanced through the lead hole 610 and the lead lumen 614, as shown in FIG. 18. The plunger 604 can be locked or maintained in this position by insertion of a locking rod 620 or other device through the aligned locking hole 612 and the locking lumen 616. Such a locking rod 620 holds the position of the plunger 606 in relation to the anchor body 602 while the spring 606 is in tension. This feature is useful when advancing the anchor 600 along a lead 100 to a desired placement position since the lead 100 is able to move freely through the lead lumen 614 in this actuated configuration.

Once the anchor 600 is disposed at its desired placement position along the lead 100, the anchor 600 is then reverted to the relaxed position, as illustrated in FIG. 19. If a locking rod 620 is present, the locking rod 620 is removed from the locking lumen 616 and locking hole 612 so that the spring 606 is able to recoil. Otherwise, if no locking rod 620 is used, pressure applied to the plunger 604 is simply released to allow the spring 606 to recoil. The spring 606, in turn, pushes the plunger 604 back out from its advanced position within the anchor body 602. This shifts the lead lumen 614 so that the lead lumen 614 is misaligned with the lead hole 610. The portion of the lead 100 within the lead lumen 614 is shifted along with the plunger 604, as illustrated in FIG. 19, creating a tortuous path for the lead 100. This tortuous path creates sufficient friction so as to hold the anchor 600 in place in relation to the lead 100.

It may be appreciated that in alternative embodiments the holes 610, 612 and lumens 614, 616 are aligned when the anchor 600 is in a relaxed position. In such embodiments, the plunger 604 is moved to shift the lead 100 into the tortuous path and the shift is maintained in the actuated position by a latch, snap, button, ridge or other feature.

### Cam Anchor

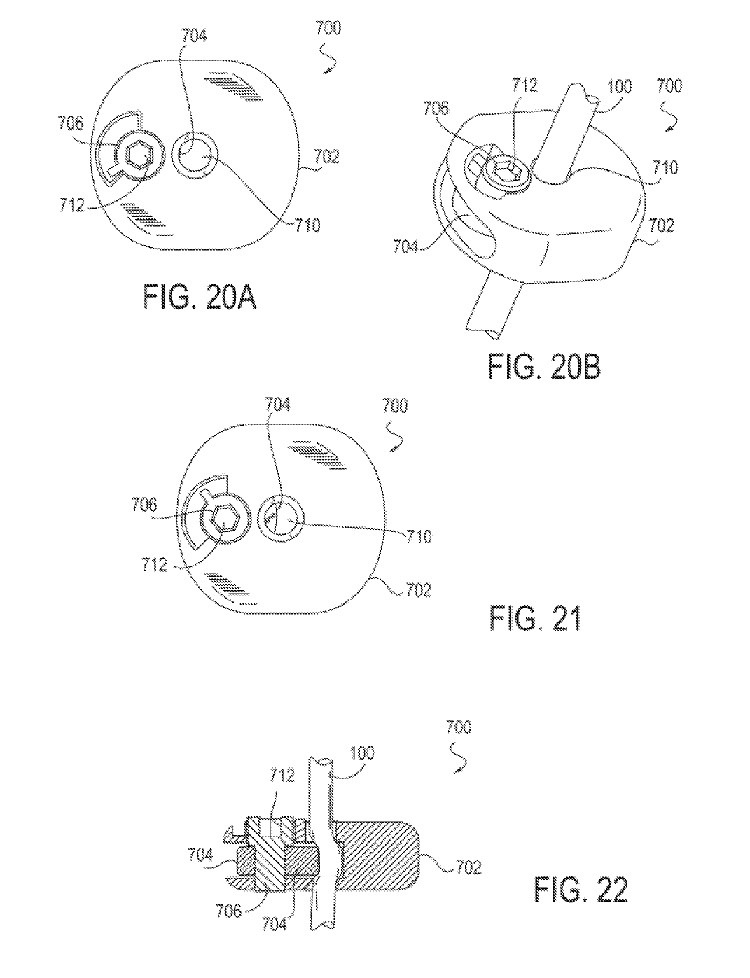

FIGS. 20A-20B, 21, 22 illustrate an embodiment of a cam anchor 700. In this embodiment, the anchor 700 is comprised of an anchor body 702, a cam 704, and an actuator 706. In this embodiment, the anchor body 702 has a disk shape, however, it may be appreciated that the anchor body 702 can have any shape which resists movement of the anchor 700 through tissue layers. Referring to FIG. 20A, the anchor body 702 includes a lead lumen 710 passing through the anchor body 702. In this embodiment, the cam 704 is moveable between two positions, a relaxed position and an actuated position. In the relaxed position, as illustrated in FIG. 20A, the actuator 706 rotates the cam 704 so that the lumen 710 is largely unobstructed by the cam 704. This allows a lead 100 to pass through the lumen 710, as illustrated in FIG. 20B. In this position, the anchor 700 is able to move along a lead 100 to a desired placement position since the lead 100 is able to move freely through the lead lumen 710 in this relaxed configuration.

The cam 704 is rotatable with the use of a tool. In this embodiment, the tool can be inserted into an actuation socket 712 and turned to rotate the cam 704 back and forth. FIG. 21 illustrates the cam 704 rotated to the actuated position wherein the cam 704 at least partially obstructs the lumen 710. Once the anchor 700 is disposed at its desired placement position along the lead 100, the actuator 706 is then rotated to move the cam 704 into the lumen 710, as illustrated in FIG. 22. The portion of the lead 100 within the lead lumen 710 is shifted, creating a tortuous path for the lead 100. This tortuous path creates sufficient friction so as to hold the anchor 700 in place in relation to the lead 100. The anchor 700 may be released from the lead 100 by rotating the cam 704 back, removing the tortuous path and allowing the anchor 700 to be repositioned along the lead 100. The cam 704 can be locked in any desired position. In some embodiments, the cam 704 is locked by positioning the cam 704 over the center. This is achieved by rotating an offset cam 704 past the point of maximum obstruction of the lumen 710. Rotation of the cam 704 back toward its original position is resisted by the lead 100, locking the cam 704, and therefore the anchor, in place.

### Jaw Anchor

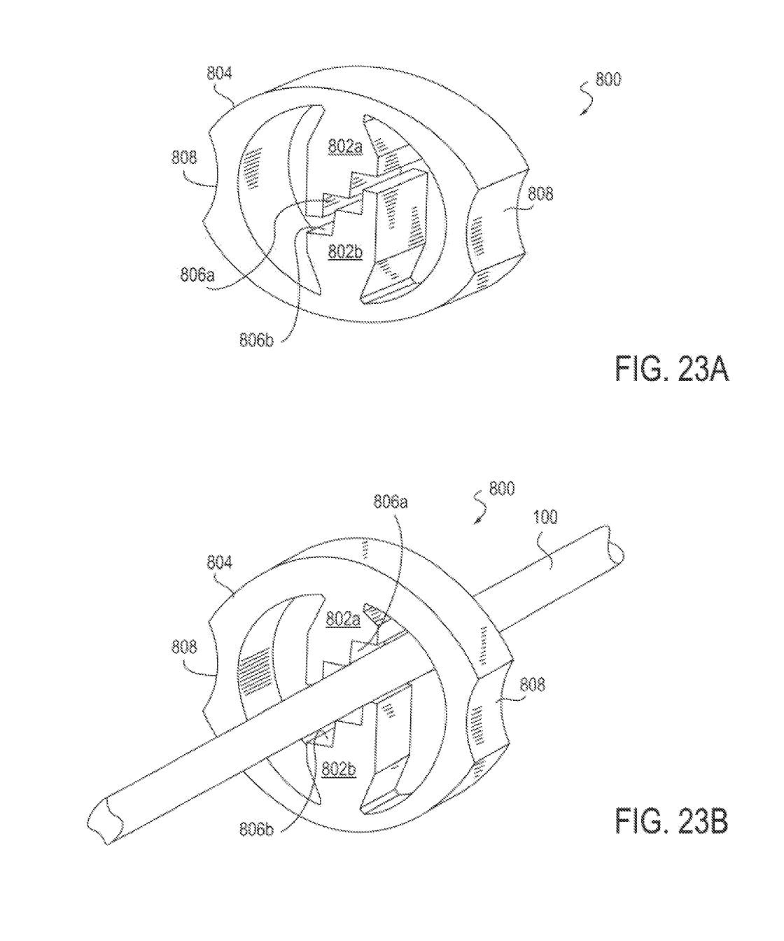

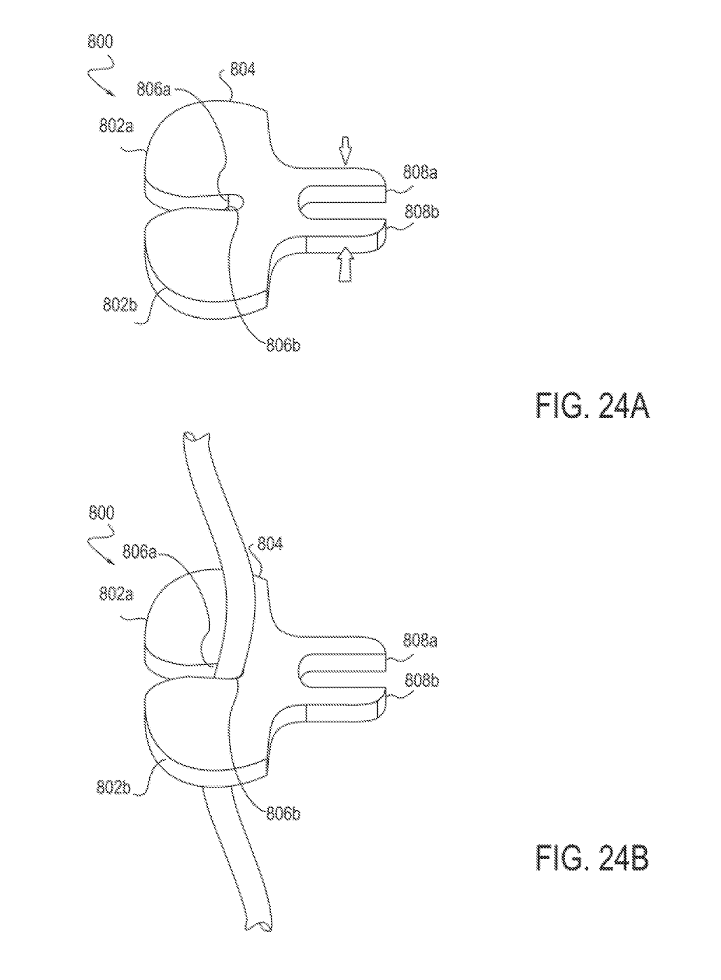

FIGS. 23A-23B, 24A-24B illustrate embodiments of a jaw anchor 800. In each embodiment, the anchor 800 includes jaws 802a, 802b which open to receive and close to secure a lead. FIGS. 23A-23B illustrate an embodiment of a jaw anchor 800 comprised of a molded body 804 which includes a first jaw 802a and a second jaw 802b. In this embodiment, the body 804 has a disk shape (FIG. 23A) and the jaws 802a, 802b are disposed in the center of the disk to receive a lead 100 in a direction perpendicular or concentric to the disk shape (FIG. 23B). In this embodiment, the first jaw 802a has a first mating surface 806a and the second jaw has a second mating surface 806b. The mating surfaces 806a, 806b are configured to fixedly hold a portion of a lead 100 therebetween when the first and second jaws 802a, 802b are mated. Typically, the lead 100 is held by friction. In some embodiments, the first mating surface 806a and/or second mating surface 806b has surface characteristics, such as curves, channels, waves, steps, grips or irregularities which increases the friction. In this embodiment, the surface characteristics comprise steps. In some embodiments the surface characteristics are matched or coordinated between the surfaces 806a, 806b as illustrated in FIG. 23A. In this embodiment, the body 804 is comprised of a flexible implantable material, such a polymer (e.g. polyetheretherketone, acrylic), silicone, metal (e.g. cobalt-chrome, stainless steel) or a composite of materials and components. By squeezing the outer perimeter of the body 804 toward its center axis of its disk shape, the body 804 flexes and moves the first jaw 802a away from the second jaw 802b so that the surfaces 806a, 806b un-mate. In some embodiments, the body 804 includes indents 808 along its perimeter to assist in positioning squeezing forces, such as from fingers or a tool. While the jaws 802a, 802b are open, a lead 100 can be passed therebetween, such as illustrated in FIG. 23B. Release of the squeezing forces from the body 804 allows the jaws 802, 802b to move toward each other so that the mating surfaces 806a, 806b engage at least a portion of the lead 100, fixedly attaching the anchor 800 to the lead 100.

The anchor 800 may be repositioned along the lead 100 by simply re-squeezing the outer perimeter of the body 804 to open the jaws 802a, 802b. The anchor 800 can then be moved relative to the lead 100 to a new desired location. Release of the outer perimeter closes the jaws 802a, 802b to fixedly attach the anchor 800 at the new desired location.

FIGS. 24A-24B illustrate another embodiment of a jaw anchor 800 comprised of a molded body 804 which includes a first jaw 802a and a second jaw 802b. In this embodiment, the body 804 has a disk shape and the jaws 802a, 802b are disposed along an edge of the disk to receive a lead 100 in a direction perpendicular or concentric to the disk shape. In this embodiment, the first jaw 802a has a first mating surface 806a and the second jaw has a second mating surface 806b. The mating surfaces 806a, 806b are configured to fixedly hold a portion of a lead 100 therebetween when the first and second jaws 802a, 802b are mated. Typically, the lead 100 is held by friction. In some embodiments, the first mating surface 806a and/or second mating surface 806b has surface characteristics, such as curves, channels, waves, steps, grips or irregularities which increases the friction. In some embodiments the surface characteristics are matched or coordinated between the surfaces 806a, 806b. In this embodiment, the body 804 is comprised of a flexible implantable material, such a polymer (e.g. polyetheretherketone, acrylic), silicone, metal (e.g. cobalt-chrome, stainless steel) or a composite of materials and components. By squeezing (as indicated by arrows) a pair of tangs 808a, 808b disposed opposite the jaws 802a, 802b, movement of the tangs 808a, 808b toward each other causes the jaws 802a, 802b to move apart. Thus, the flexible material is flexible enough to allow flexing of the body 804 yet rigid enough to translate the force from the tangs 808a, 808b to the jaws 802a, 802b. It may be appreciated that the tangs 808a, 808b may be squeezed by fingers or with the use of a tool. While the jaws 802a, 802b are open, a lead 100 can be passed therebetween, such as illustrated in FIG. 24B. Release of the squeezing forces from the tangs 808a, 808b allows the jaws 802, 802b to move toward each other so that the mating surfaces 806a, 806b engage at least a portion of the lead 100, fixedly attaching the anchor 800 to the lead 100.

The anchor 800 may be repositioned along the lead 100 by simply re-squeezing the tangs 808a, 808b to open the jaws 802a, 802b. The anchor 800 can then be moved relative to the lead 100 to a new desired location. Release of the tangs 808a, 808b closes the jaws 802a, 802b to fixedly attach the anchor 800 at the new desired location.

It may be appreciated that the jaw anchor 800 of FIGS. 24A-24B can be attached and detached from a lead 100 at any location along the lead body due to the side opening of the jaws 802a, 802b. Thus, it can be attached or detached to a lead wherein its ends are not accessible. In contrast, the jaw anchor 800 of FIGS. 23A-23B is advanceable from one end of a lead body to its other end to position the anchor 800 at any location therealong due to the center opening of the jaws 802a, 802b.

It may also be appreciated that, in some embodiments, actuation of the jaws 802a, 802b is achieved with the use of a spring rather than the flexure properties of the anchor body.

### Flapper Anchor

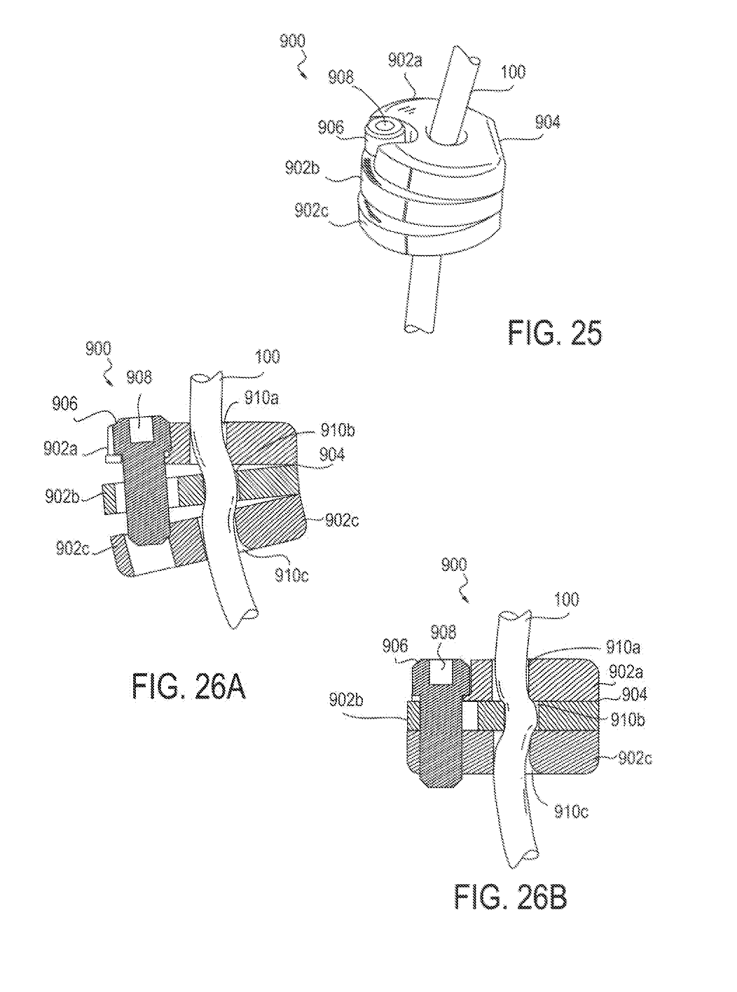

FIG. 25 illustrates an embodiment of a flapper anchor 900. In this embodiment, the anchor 900 is comprised of three plates or flaps 902a, 902b, 902c, however it may be appreciated that flapper anchors 900 have at least two flaps. The flaps 902a, 902b, 902c are connected to each other on one edge, such as by a hinge 904. An actuator 906 passes through at least a portion of each flap 902a, 902b, 902c, wherein movement of the actuator 906 brings the flaps closer together or farther apart. In this embodiment, such movement is achieved by rotation of the actuator 906 back and forth. Typically, a tool is inserted into an actuation socket 908 in the actuator 906 and rotated to rotate the actuator 906.

Each flap 902a, 902b, 902c has a lead lumen 910a, 910b, 910c, respectively, passing therethrough. The lead lumens 910a, 910b, 910c are arranged so that the lumens 910a, 910b, 910c are at least aligned so that a lead 100 can pass through each of the lumens 910a, 910b, 910c while the anchor is in an open position, as illustrated in FIG. 26A. Thus, the anchor 900 is moveable along the lead 100 when in the open position. Once the anchor 900 is disposed at its desired placement position along the lead 100, the actuator 906 is then rotated to move the flaps 902a, 902b, 902c toward each other. The flaps 902a, 902b, 902c move together so that they mate surface to surface forming a closed position. In the closed position, the lumens 910a, 910b, 910c are misaligned creating a tortuous path, as illustrated in FIG. 26B. The lead 100 passing therethrough is forced into the tortuous path. This tortuous path creates sufficient friction so as to hold the anchor 900 in place in relation to the lead 100. The anchor 900 may be released from the lead 100 by rotating the actuator 906 back, separating the flaps 902a, 902b, 902c and thus removing the tortuous path. This allows the anchor 900 to be repositioned along the lead 100. The actuator 906 can be locked in any desired position.

### Balloon Anchor

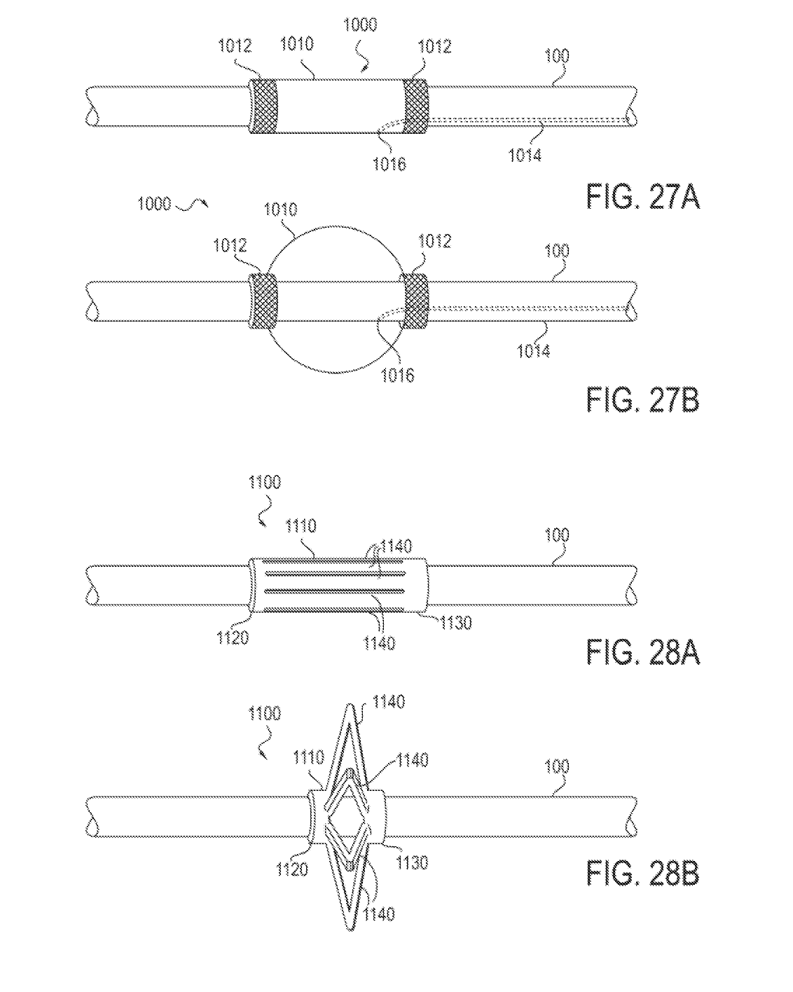

FIGS. 27A-27B illustrate an embodiment of a balloon anchor 1000. In this embodiment, the balloon anchor 1000 is comprised of a flexible sheath 1010 that is mounted on a lead 100 (or other device that is to be anchored) to form a balloon cuff. Typically, the sheath 1010 is adhered to the body of the lead 100 with adhesive 1012, such as a UV curable adhesive. Alternatively, the sheath may be held in place by rings which are crimped over the ends of the sheath 1010. The lead 100 includes at least one lumen 1014 extending to the balloon anchor 1000 to deliver inflation medium to the anchor 1000 through a port 1016. Delivery of inflation medium inflates the anchor 1000 to a desirable size, as illustrated in FIG. 27B. Thus, the size of the anchor 1000 can be adjusted to suit the implantation environment.

In some embodiments, the flexible sheath 1010 is comprised of a polymer material, such as silicone, polyethylene terephthalate, nylon, polyurethane, or other medical device balloon materials. In some embodiments, the inflation medium is comprised of saline. In other embodiments, the inflation medium is comprised of a material that hardens once delivered, such as polymethylmethacrylate (PMMA). In such embodiments, a two part formulation may be mixed in an injection syringe and injected in an uncured form. The material would then cure in place (i.e. in situ) over time.

It may be appreciated that in the embodiment of FIGS. 27A-27B, the balloon anchor 1000 is fixedly attached to the lead 100 so that it is not advanceble along the lead body. In other embodiments, the balloon anchor 1000 is separate from the lead 100 to allow advancement along the lead body for position adjustment.

### Umbrella Anchor

FIGS. 28A-28B illustrate an embodiment of an umbrella anchor 1100. In this embodiment, the umbrella anchor 1100 is comprised of a sheath 1110 that is mountable on a lead 100 (or other device that is to be anchored), as illustrated in FIG. 28A. The sheath 1110 has a first end 1120, a second end 1130 and a plurality of slats 1140 such that movement of the first end 1120 and the second end 1130 toward each other causes at least some of the slats 1140 to bend and protrude outward, as illustrated in FIG. 28B. The protruding slats 1140 form create a disk shape which resists movement through tissue layers when placed between layers as described herein above. The slats 1140 may be of any suitable length to create a disk having the desired dimension. In some embodiments, the sheath 1110 is comprised of a flexible material, such as a polymer material (e.g. silicone, polyethylene terephthalate, nylon, polyurethane, or the like). In other embodiments, the sheath 1110 is comprised of a rigid or semi-rigid material having flex points to allow the slats 1140 to bend outwardly.

In some embodiments, the first end 1120 of the sheath 1110 is adhered to the body of the lead 100 with adhesive 1012, such as a UV curable adhesive. Thus, the second end 1130 is free to move to a desired location. The second end 1130 is then adhered in place after actuation of the anchor 1100, such as with the use of a ring which is crimped over the second end 1130. Alternatively, both ends 1120, 1130 may be fixed to the lead 100 in situ, such as with crimping rings.

It may be appreciated that in some embodiments, the umbrella anchor 1100 may be formed in the actuated position and advanced over the lead 100 to a desired position. At such position, the anchor 110 may then be fixed to the lead 100, such as with crimping rings.

It may be appreciated that both the balloon anchor 1010 and the umbrella anchor 1100 provide a low profile anchor during delivery. This reduces the size of the incision in the tissue layer needed to insert the anchor. In some embodiments, no suturing is needed to close the incision since any opening in the tissue layer is filled with the lead 100.

### Twist-Grip Anchor

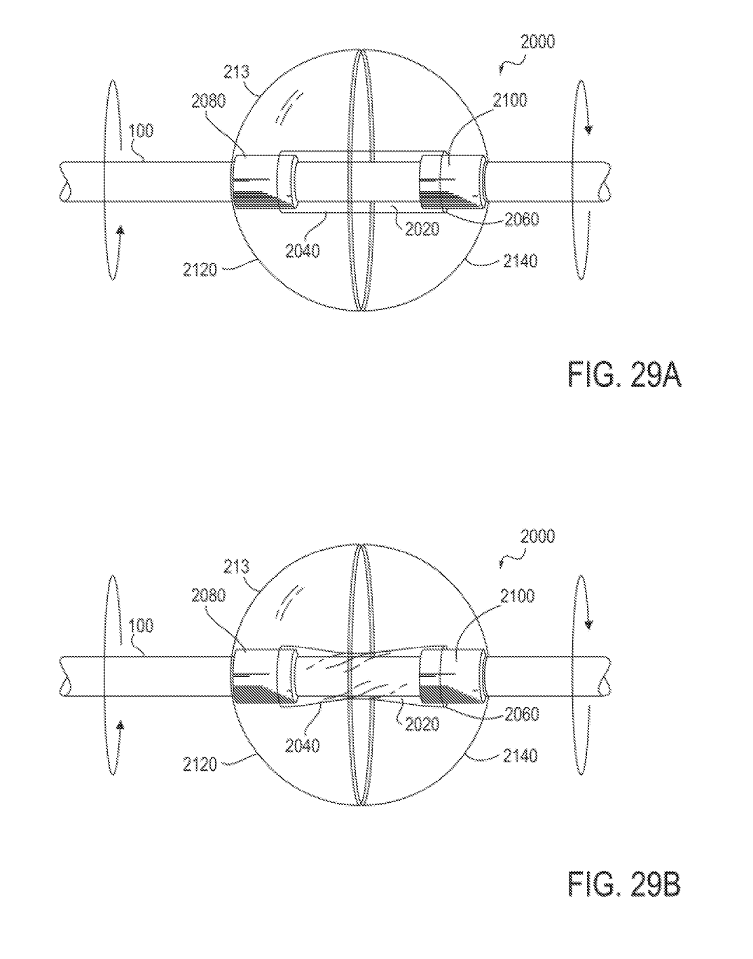

FIGS. 29A-29B illustrate an embodiment of a twist-grip anchor 2000. In this embodiment, the anchor 2000 is comprised of an inner sleeve 2020 having a first end 2040 and a second end 2060. The inner sleeve 2020 is comprised of an implantable flexible or semi-flexible material, such as silicone, polyurethane, silicone-urethane copolymers or other suitable materials. The first end 2040 is fixedly attached to a first support 2080 and the second end 2060 is fixedly attached to a second support 2100. The supports 2080, 2100 are comprised of a more rigid material which sufficiently maintains the inner diameter of the supports 2080, 2100 during actuation of the anchor 2000. Example materials include polyetheretherketone, implantable acrylic, and stainless steel. The anchor 2000 also includes a rotatable two-piece outer housing 2130 comprised of a first piece 2120 and a second piece 2140. The first piece 2120 is fixedly attached to the first end 2040 and the second piece 2140 is fixedly attached to the second end 2060. In this embodiment, the first and second pieces 2120, 2140 extend over the inner sleeve 2020 so that the inner sleeve 2020 is encased by the housing 2130. Typically, the first and second pieces 2120, 2140 mate at a location over the inner sleeve, such as in the center of the sleeve, as illustrated in FIG. 29A. It may be appreciated, however, that the pieces 2120, 2140 may mate at other locations or may not mate at all.

In some embodiments, the first and second pieces 2120, 2140 are circumferentially rotatable in opposite directions relative to each other around a central axis. In other embodiments, the first piece 2120 is stationary and the second piece 2140 rotates in relation to the first piece 2120. Once rotated, the first and second pieces 2120, 2140 are offset from each other by, for example, up to 360 degrees, up to 270 degrees, up to 180 degrees, up to 90 degrees, up to 45 degrees, or less than 45 degrees. In preferred embodiments, the pieces 2120, 2140 are offset from each other by 90-180 degrees. In other embodiments, the pieces 2120, 2140 are rotatable in increments, such as in 10 degree increments. In any case, rotation offsets the first end 2040 of the inner sleeve 2020 relative to the second end 2060 of the inner sleeve. This causes the inner sleeve 2020 to twist and collapse. The outer housing 2130 includes a locking mechanism which locks the first and second pieces 2120, 2140 together. Thus, the first and second pieces 2120, 2140 can be rotated relative to each other and locked in the rotated position. This holds the sleeve 2020 in the twisted position.

FIG. 29A illustrates the embodiment of the twist-grip anchor 2000 mounted on a lead 100. The anchor 2000 is advanceable along the lead 100 to a desired location for anchoring the lead 100 to surrounding tissue. Once desirably positioned, the anchor 2000 is fixedly attached to the lead 100 by actuating the anchor 2000. Actuation is achieved by rotating the first and/or second pieces 2120, 2140 relative to each other. This causes the inner sleeve 2020 to twist and collapse against the lead 100, as illustrated in FIG. 29B. Such collapse, along with the sleeve friction on the lead 100, retains the lead 100 axially and thus fixedly attaches the anchor 2000 to the lead 100. Since the inner sleeve 2020 is compliant, the sleeve 2020 conforms to the lead 100 in an atraumatic manner which resists damage to the lead 100 and the anchor 2000, even under conditions of motion fatigue. In some embodiments, twisting and collapse of the inner sleeve 2020 causes slight deformation of the lead 100, particularly if the lead 100 is flexible. This assists in retaining the lead 100 and does so in a flexible manner, again resisting damage to the lead 100 and the anchor 2000. Thus, the twist-grip anchor 2000 is particularly suited for use with flexible leads which are typically difficult to retain without damage when using conventional anchors. The level of grip on the lead 100 can be adjusted by increasing or decreasing the amount of twist (i.e. by increasing or decreasing rotation of the first and/or second pieces 2120, 2140 relative to each other.

Once the desired level of grip is achieved, the pieces 2120, 2140 are locked in relation to each other to maintain the rotation. Such locking is achieved with a locking mechanism, such as a one-way ratchet with spring loading, a clutch arrangement, a cam and/or a plunger lock. In some embodiments, the locking mechanism is operated with the use of a tool, and in other embodiments the locking mechanism is operated by hand.

The anchor 2000 can be disengaged or removed from the lead 100 by unlocking the locking mechanism and untwisting the inner sleeve 202. This is achieved by reversing the rotation of the relevant pieces 2120, 2140. The anchor 2000 can then be repositioned and reengaged at a new desired location along the lead 100. However, in some embodiments, the locking mechanism is a one-time use wherein repositioning or removal of the anchor 2000 involves clipping off or removing the locking mechanism. In such instances, if repositioning is desired, a new locking mechanism is attached to the anchor 2000 or a new anchor having an intact locking mechanism is used.

It may be appreciated that the twist-grip anchor 2000 may be biased to twist and collapse against a lead 100 while in a relaxed state, wherein actuation opens the lumen of the inner sleeve 2020 to allow advancement of the lead 100 therein. In such embodiments, the locking mechanism locks the first and second pieces 2120, 2140 together in an unrotated, non-offset or aligned position. This allows the anchor 2000 to be advanced along the lead 100. Once desirably placed, the locking mechanism may be disengaged or unlocked to allow the pieces 2120, 2140 to return to a biased rotation, twisting the inner sleeve 2020 against the lead 100.

It may be appreciated that in some embodiments, the anchor 2000 includes more than one inner sleeve. For example, the anchor 2000 may have two inner sleeves. Such inner sleeves are axially aligned so that a lead 100 is passable through each of the sleeves. In this embodiment, the first inner sleeve has a first end and a second end and the second inner sleeve has a first end and a second end. The anchor 2000 includes three supports. The first end of the first sleeve is fixedly attached to the first support and the second end is fixedly attached to a second support. The first end of the second sleeve is fixedly attached to the second support and the second end is fixedly attached to the third support. The supports are comprised of a more rigid material which sufficiently maintains the inner diameter of the supports during actuation of the anchor 2000.

In this embodiment, the anchor 2000 also includes a rotatable three-piece outer housing comprised of a first piece 2120, a second piece 2140, such as those illustrated in FIGS. 29A-29B, and a third piece such as a band therebetween. The first piece is fixedly attached to the first support, the second piece is fixedly attached to the second support, and the third piece is fixedly attached to the third support. In this embodiment, the first and second pieces extend over the inner first inner sleeve and mate at a location over the first inner sleeve, such as in the center of the sleeve. And, in this embodiment, the second and third pieces extend over the second inner sleeve and mate and a location over the second inner sleeve, such as in the center of the sleeve. The first and second pieces are circumferentially rotatable in opposite directions relative to each other around a central axis. And, the second and third pieces are circumferentially rotatable in opposite directions relative to each other around the same central axis. In other embodiments, the second piece is stationary while the first piece and third piece rotate in relation to the second piece. Rotation of some or all of the pieces causes the inner sleeves to twist and collapse against the lead 100. It may be appreciated that the pieces may be independently rotatable or some or all of the pieces may rotate together. The outer housing includes at least one locking mechanism which locks the pieces together.

It may also be appreciated that in some embodiments, the inner sleeve 2020 is comprised of a rigid material. In such embodiments, the sleeve 2020 is comprised of a tube having geometries, such as preferential cuts or cut-outs, which collapse around the lead 100 in a predetermined fashion when twisted. In some embodiments, the sleeve 2020 includes cuts in a spiral arrangement which cause the sleeve 2020 to collapse inward when rotated in one direction and extend outward when rotated in the opposite direction. Such collapse engages the sleeve with the lead and extension disengages the sleeve from the lead. In some embodiments, angled cuts around the circumference of the sleeve provide a similar benefit.

It may also be appreciated that each of the above mentioned anchor designs may be comprised partially or wholly of a material which allows or encourages tissue ingrowth. Examples of such materials include a fabric, netting or screen. Alternatively or in addition, the anchor may include a surface geometry or texture which allows or encourages tissue ingrowth. In any case, such tissue ingrowth may assist in stabilizing the anchor and maintaining position of the anchor within the patient's body.

### Anchor Features

It may be appreciated that each of the above mentioned anchor designs may have a variety of overall shapes. Although some embodiments are depicted as disks, cylinders or spheres, each embodiment is not limited to the shapes illustrated in the example embodiments. For instance, an anchor having a disk shape may alternatively have a sphere shape by adding some additional body material to form a sphere shape while maintaining the basic features of the anchor, particularly the features which provide attachment to a lead. Likewise, a sphere shape may be modified into a disk shape by removing some body material. In any case, the overall shape of the anchor atraumatically resists movement through adjacent tissue layers.

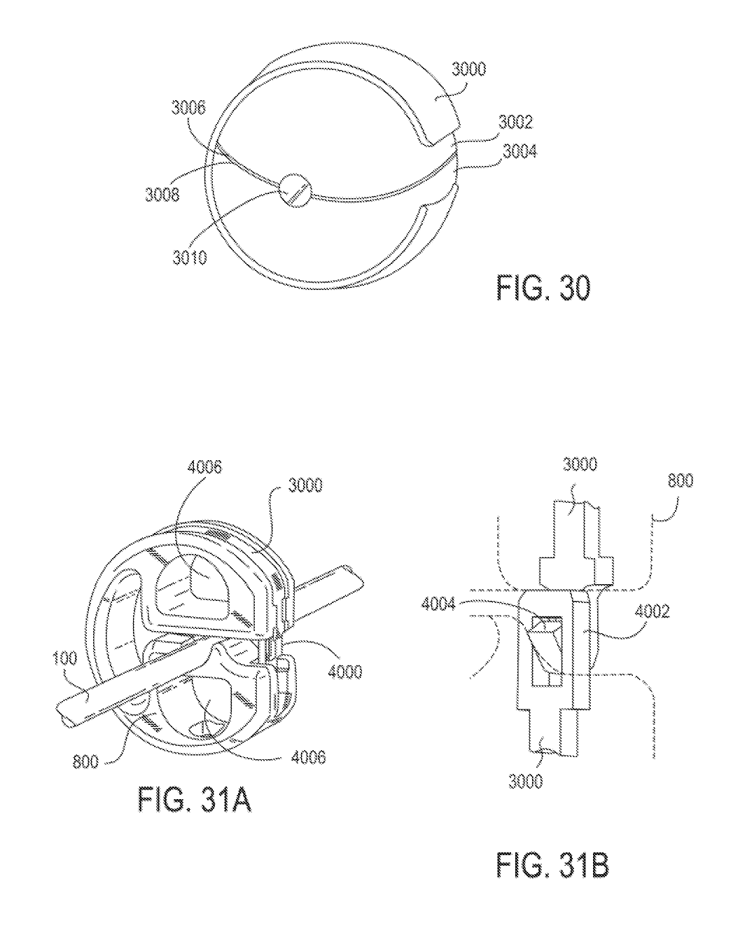

It may be appreciated that each of the above mentioned anchor designs may be supported by a closure device 3000. An example embodiment of a closure device 3000 is illustrated in FIG. 30. In this embodiment, the tissue-captured anchor 200 is comprised of a first portion 3002 and second portion 3004 which are mateable. It may be appreciated that the first portion 3002 has a first mating surface 3006 and the second portion 3004 has a second mating surface 3008, wherein mating of the first and second surfaces together attaches the anchor 200 to an elongate device (not shown), such as like a wave anchor described above. Thus, the mated surfaces 3006, 3008 form a lumen 3010 therethrough, through which the elongate device passes. The closure device 3000 is positionable around the anchor 200, such as at least sufficiently around the anchor 200 to assist in holding the first and second mating surfaces 3006, 3008 in contact. In this embodiment, the closure device 3000 has a “C” shape which fits at least partially around the perimeter of the anchor 200 which has a ball shape. The closure device acts as a spring and applies force to the at least partially encircled anchor to assist in holding the mated configuration. In some embodiments, the closure device also compresses the anchor onto the elongate device to which it is attached. In some embodiments, the closure device 3000 is comprised of a flexible metal, such as a stainless steel, a shape-memory metal, nitinol, etc. In other embodiments, the closure device 3000 is comprised of a polymer. The polymer may be at least semi-rigid so that the closure device acts like a clamp, or the polymer may be flexible so that the closure device acts like a rubber band. In any case, the closure device may be removed from the anchor by applying force to pull the closure device away from the anchor. Or, the anchor may be removed from the elongate device while the anchor and closure device is attached thereto by wedging a tool between the first and second mating surfaces 3006, 3008, thereby releasing the elongate device.

In some embodiments, the closure device 3000 further includes a locking device 4000. FIGS. 31A-31B illustrate an embodiment of a locking device 4000 used with the closure device 3000 of FIG. 30. FIG. 31A shows the closure device 3000 used with an embodiment of a jaw anchor 800. Here, the closure device 3000 has a “C” shape, wherein the ends of the “C” shape are fixable together by the locking device 4000. Thus, the closure device 3000 may assist in holding the jaw anchor closed, and the locking device 4000 may further assist by holding the closure device closed. In this embodiment, the locking device 4000 is comprised of a latch 4002, illustrated enlarged in FIG. 31B. As shown, the latch 4002 is moveable between a locked and unlocked configuration by moving a switch 4004. Typically, such movement is achieved with the use of a tool due to its small size. To remove the closure device, the latch 4002 is moved to the unlocked position and the closure device may be removed from the anchor by applying force to pull the closure device away from the anchor. Or, in this embodiment, the anchor may be removed from the elongate device while the anchor and closure device is attached thereto by positioning a tool within holes 4006 in the jaw anchor to assist in separating the jaws, thereby releasing the elongate device.

It may also be appreciated that each of the above mentioned anchor designs may be comprised partially or wholly of a material which allows or encourages tissue ingrowth. Examples of such materials include a fabric, netting or screen. Alternatively or in addition, the anchor may include a surface geometry or texture which allows or encourages tissue ingrowth. In any case, such tissue ingrowth may assist in stabilizing the anchor and maintaining position of the anchor within the patient's body.

It may also be appreciated that each of the above mentioned anchor designs may be fixedly or removably attached to a lead or other device. Alternatively or in addition, the lead or device may be looped, knotted or threaded through the anchor to maintain position of the anchor in relation to the lead.

It may also be appreciated that in each of the above mentioned anchor designs, the anchor may be held between the tissue layers by the anchor alone or in combination with adhesive or suturing of the anchor to any of the surrounding tissue.

It may be appreciated that each of the above mentioned anchor designs may be used to anchor a variety of devices. Although the above anchor embodiments are described to be attached to leads, such anchors may be attached to any suitable device that is at least partially implantable. Examples of such devices include catheters, scopes, needles, cannulas or any tube-like structure regardless of cross-sectional geometry.

Although the foregoing invention has been described in some detail by way of illustration and example, for purposes of clarity of understanding, it will be obvious that various alternatives, modifications, and equivalents may be used and the above description should not be taken as limiting in scope of the invention which is defined by the appended claims.

1. An anchor for anchoring an elongate device within a body of a patient comprising:
an anchor body suturelessly attachable to the elongate device at an anchoring point and contoured so as to
a) be positionable between a first tissue layer and a second tissue layer within the body while the elongate device passes through the first and second tissue layers, and
b) atraumatically resist movement through the tissue layers thereby anchoring the elongate device between the tissues at the anchoring point.

2. An anchor as in claim 1, wherein the contour has a ball, round, elliptical, oval, oblong or disk shape.

3. An anchor as in claim 1, wherein the anchor has a diameter of less than 0.5 inches.

4. An anchor as in claim 1, wherein the anchor is sized and contoured to be passable through an incision in a muscle or ligament, wherein the incision has a length of 1 inch or less.

5. An anchor as in claim 1, wherein the contour includes at least one protruding portion which extends laterally outward from the elongate device.

6. An anchor as in claim 1, wherein the anchor body comprises a first portion having first lumen configured for passage of the elongate device therethrough and a second portion having a second lumen configured for passage of the elongate device therethrough, wherein the first and second lumens are alignable for passage of the elongate device therethrough.

7. An anchor as in claim 6, wherein a misalignment of the first and second lumens attaches the anchor body to the elongate device.

8. An anchor as in claim 7, wherein the first portion comprises a plunger which is advanceable within the second portion so such advancement aligns or misaligns the first and second lumens.

9. An anchor as in claim 7, wherein alignment or misalignment is maintained by force of a spring.

10. An anchor as in claim 7, wherein the first portion is moveable toward the second portion wherein such movement causes the lumens to misalign.

11. An anchor as in claim 6, wherein the first portion has a first mating surface and the second portion has a second mating surface, wherein mating of the first and second surfaces together attaches the anchor to the elongate device.

12. An anchor as in claim 11, wherein the first portion has a protrusion through which the first lumen passes and the second portion has a recession through which the second lumen passes, wherein mating of the protrusion with the recession aligns the first and second lumens and attaches the anchor to the elongate device.

13. An anchor as in claim 6, wherein the first portion has a first perimeter and the second portion has a second perimeter wherein aligning or misaligning the perimeters attaches the anchor to the elongate device.

14. An anchor as in claim 6, wherein rotating the first portion in relation to the second portion attaches the anchor to the elongate device.

15. An anchor as in claim 1, wherein the anchor body comprises a first mating surface and a second mating surface, wherein the first and second mating surfaces are mateable to each other while the elongate device is disposed therebetween.

16. An anchor as in claim 15, wherein the anchor body comprises a first portion having the first mating surface and a separate second section having the second mating portion, wherein the first and second portions are joinable.

17. An anchor as in claim 15, wherein the first mating surface is disposed on a first jaw and the second mating portion is disposed on a second jaw, wherein the jaws are connected on at least one side and open to receive the elongate device therebetween.

18. An anchor as in claim 17, wherein the anchor body is configured so that squeezing an outer perimeter of the anchor body toward its center axis flexes and moves the first jaw away from the second jaw so that the surfaces un-mate.

19. An anchor as in claim 17, wherein the first and second jaws form a side opening in the anchor body for insertion of the elongate device therebetween.

20. An anchor as in claim 1, wherein the anchor body has a lumen configured for passage of the elongate device therethrough and a cam arranged to at least partially obstruct the lumen so as to attach the anchor to the elongate device.

21. An anchor as in claim 1, wherein the anchor body is removeably attachable to the elongate device.

22. A method for anchoring an elongate device within a body of a patient comprising:
positioning an anchor between a first tissue layer and an adjacent second tissue layer within the body; and
suturelessly attaching the anchor to an elongate device at an anchoring point, wherein the elongate device is positioned through the first tissue layer and the second tissue layer within the body and wherein the anchor is contoured to atraumatically resist movement through the tissue layers thereby anchoring the elongate device between the tissues at the anchoring point.

23. A method as in claim 22, wherein positioning comprises positioning the anchor laterally adaj cent to a spinous process.

24. A method as in claim 23, wherein first tissue layer comprises a spinous muscle layer.

25. A method as in claim 24, wherein the second tissue layer comprises a spinous muscle layer.

26. A method as in claim 22, wherein the first or second layer comprises fascia, a spinae erector, an illiocostalis lumborum, a longissimus thoriclis, a longissimus cervicus, an illioconstalis cervicis, a serratus anterior, a ligament, a supraspinous ligament, an interspinous ligament, a ligamentum flavum, an alar ligament, an anterior atlantoaxial ligament, a posterior atlantoaxial ligament, a ligamentum nuchae, an anterior longitudinal ligament, a posterior longitudinal ligament, an interspinous ligament, an intertransverse ligament, an iliolumbar ligament, a sacroiliac ligament, a sacrospinous ligament, a sacrotuberous ligament, an anterior occipitoatlantal ligament, a posterior occipitoatlantal ligament, a lateral occipitoatlantal ligament, an occipitoaxial ligament, an apical ligament, an altantoaxial ligament, a lateral ligament, a transverse ligament, a superior longitudinal fascicle, an inferior longitudinal fascicle, an aponeurosis, a tendon, a subcutaneous tissue, skin, a dermal layer, a bone, cartilage, or an artificial tissue.

27. A method as in claim 22, wherein the anchor body comprises a first portion having a first lumen configured for passage of the elongate device therethrough and a second portion having a second lumen configured for passage of the elongate device therethrough, the method further comprising mounting the anchor on the elongate device by passing the elongate device through the first and second lumens while the lumens are aligned.

28. A method as in claim 27, wherein suturelessly attaching the anchor to the elongate device comprises misaligning the lumens

29. A method as in claim 27, wherein the first portion has a first mating surface and the second portion has a second mating surface, wherein suturelessly attaching the anchor to the elongate device comprises mating the first and second surfaces together.

30. A method as in claim 27, wherein the first portion has a first perimeter and the second portion has a second perimeter, wherein suturelessly attaching the anchor to the elongate device comprises aligning or misaligning the perimeters.

31. A method as in claim 22, wherein the anchor body comprises a first mating surface and a second mating surface, wherein suturelessly attaching the anchor to the elongate device comprises mating the first and second mating surfaces to each other while the elongate device is disposed therebetween.

32. A method as in claim 31, wherein the first mating surface is disposed on a first jaw and the second mating surface is disposed on a second jaw, wherein suturelessly attaching the anchor to the elongate device comprises opening the jaws to receive the elongate device therebetween.

33. A method as in claim 32, wherein opening the jaws comprises squeezing an outer perimeter of the anchor body toward its center axis which causes the first jaw to move away from the second jaw

34. A method as in claim 32, wherein the first and second jaws form a side opening in the anchor body, and wherein suturelessly attaching the anchor to the elongate device comprises inserting the elongate device into the side opening.

35. A method as in claim 22, further comprising releasing the attachment of the anchor to the elongate device.

36. A method as in claim 35, further comprising suturelessly re-attaching the anchor to the elongate device.
